(19) United States
(12) Patent Application Publication
(10) Pub. No.: US 2003/0203045 A1
(43) Pub. Date: Oct. 30, 2003

(54) Therapeutic composition for the treatment of HIV-1 and HIV-2

(76) Inventor: Boguslaw Jelen, Plizno (PL)

Correspondence Address: Michael R. McKenna, Suite 3800, 500 West Madison, Chicago, IL, 60661, US

(21) Appl. No.: 10/353,483

(22) Filed: Jan. 29, 2003

(30) Foreign Application Priority Data
Apr. 30, 2002 (PL) P353693

(51) Int. Cl. A61K 38/17; A61K 31/715; A61K 31/513; A61K 33/36; A61K 33/24; A61K 33/32; A61K 33/38

(52) U.S. Cl. 424/618; 424/643; 424/655; 514/2; 514/54; 514/355; 514/269; 424/670

(57) ABSTRACT

A pharmaceutical mixture and a method of its production of a therapeutic composition for blocking the HIV-1 and HIV-2 virus replication in the CD4+ cells of the human immune system in all stages of that viral infection, and in AIDS for the treatment of HIV in a patient in need of such treatment.

### BACKGROUND OF THE INVENTION

A pharmaceutical mixture for blocking the HIV-1 and/or HIV-2 virus replication in the CD4+ cells of the human immune system in all stages of that viral infection, and in AIDS, also the method of production of the pharmaceutical mixture for blocking the HIV-1 and HIV-2 virus replication in the CD4+ cells of the human immune system in all stages of that viral infection, and in AIDS.

Hereinafter it should be understood that the terms “HIV treatment” and “treatment of HIV in a patient in need of such treatment” mean treating a patient with HIV-1 and/or HIV-2. Additionally, the term “blocking HIV virus replication” means blocking HIV-1 and/or HIV-2 virus replication. Further the term “HIV virus” refers to the HIV-1 and/or HIV-2 virus. Still further, the term “HIV infection” refers to HIV-1 and/or HIV-2 infection.

The subject of the invention is a pharmaceutical mixture for blocking the HIV-1 and HIV-2 virus replication in the CD4+ cells of the human immune system in all stages of that viral infection, and in AIDS, also the method of production of the pharmaceutical mixture for blocking the HIV-1 and HIV-2 virus replication in the CD4+ cells of the human immune system in all stages of that viral infection, and in AIDS.

The invented mixture has a particular application in treatment of the Acquired Immunodeficiency Syndrome, or AIDS.

Synopsis of the Description

The subject of the invention is a pharmaceutical mixture for blocking the HIV-1 and HIV-2 virus replication in the CD4+ cells of the human immune system in that viral infection, and in AIDS contains previously known carriers and/or auxiliary substances, as well as active substances, and it is characterized by the presence of at least 10 chemical substances as active substances, among them, allocriptonine (enantiomer), present in the amount from 1.0 mg to 10.0 mg.; nimodipine, present in the amount from 20.0 mg to 100.0 mg.; potassium iodide, present in the amount from 120.0 mg to 560.0 mg.; potassium iodate, present in the amount from 30.0 mg to 140.0 mg.; inuline, present in the amount from 125.0 mg to 375.0 mg.; silver, present in the amount from 0.10 mg to 0.50 mg.; zinc, present in the amount from 10.0 mg to 20.0 mg.; chromium, present in the amount from 0.05 mg to 0.20 mg.; orotic acid, present in the amount from 150.0 mg to 500.0 mg.; desferrine, present in the amount from 100.0 mg to 300.0 mg.

### SUMMARY

A pharmaceutical mixture and a method of its production of a therapeutic composition for blocking the HIV-1 and HIV-2 virus replication in the CD4+ cells of the human immune system in all stages of that viral infection, and in AIDS for the treatment of HIV in a patient in need of such treatment, the therapeutic composition comprising of pharmaceutically effective amounts of allocriptonine enantiomer, nimodipine, potassium iodide, potassium iodate, inuline, silver, zinc, chromium, orotic acid and desferrine.

### DETAILED DESCRIPTION OF THE INVENTION

The invention pertains also to the method of production of the pharmaceutical mixture for blocking the HIV-1 and HIV-2 virus replication in the CD4+ cells of the human immune system in all stages of that viral infection, and in AIDS, characterized by mixing, in the room temperature, of allocnptonine (enantiomer), in the amount from 1.0 mg to 10.0 mg.; nimodipine, in the amount from 20.0 mg to 100.0 mg.; potassium iodide, in the amount from 120.0 mg to 560.0 mg.; potassium iodate, in the amount from 30.0 mg to 140.0 mg.; inuline, in the amount from 125.0 mg to 375.0 mg.; silver, in the amount from 0.10 mg to 0.50 mg.; zinc, in the amount from 10.0 mg to 20.0 mg.; chromium, in the amount from 0.05 mg to 0.20 mg.; orotic acid, in the amount from 150.0 mg to 500.0 mg.; desferrine, in the amount from 100.0 mg to 300.0 mg., until a mix approximating uniform substance is produced, subsequently pressed into tablets, and can also have a form of coated tablets, or can be shaped into a wafer, a suppository, effervescent powder, gel, or colloid solution; or made into suspension, syrup, salt soluble in body fluids, or liquid for intramuscular or IV injections, also in ampules.

In the existing publications pertaining to treatment regimens for patients with a diagnosed HIV infection, or with the Acquired Immunodeficiency Syndrome (AIDS), there has been no description of any medications/pharmaceutical preparations for blocking the HIV-1 and HIV-2 virus replication in the CD4+ cells of the human immune system based on the hypothesis adopted here.

The assumption underlying the invention discussed here is the hypothesis that the basis for the HIV-I and HIV-2 virus replication in the CD4+ cells of the human immune system is the exeitono-excimeric correlation between the HIV-I and HIV-2 viruses and the CD4 cell of the human immune system.

The effect of the adoption of that hypothesis is as follows: the initiation of the therapy aimed at treatment of the HIV-1 and HIV-2 viral infections, and AIDS, is reduced to delivering substances completely blocking the mechanism causing the HIV-1 and HIV-2 replication to the patient's body.

The following substances proved to be helpful in that task: allocriptonine, an alkaloid found in the Chelidonium maius plant, nimodipine, potassium iodide, potassium iodate, inuline, silver, zinc, chromium, orotic acid and desferrine.

It is known that: allocriptonine is a nucleotide phosphodiesterase blocker, nimodipine is a calcium channels blocker, potassium iodide when combined with potassium iodate in the acidic environment, produces iodic free radicals, inuline is a polisaccharide which is not decomposed in the body; silver, zinc, and chromium are elements playing a role in the enzymatic processes,—desferrine cholinates ferric ions,—orotic acid a the precursor of the nucleotide compounds.

However, the substances listed above have never appeared nor been applied together, and in particular they have not been used together in the therapeutic application such as those described in this invention.

The goal of the invention is supplying the pharmaceutical mixture for blocking the HIV-1 and HIV-2 virus replication in the CD4+ cells of the human immune system.

The invention provides that the pharmaceutical mixture for blocking the HIV-1 and HIV2 virus replication in the CD4+ cells of the human immune system in all stages of that viral infection, and in AIDS, contains previously known carriers and/or auxiliary substances, as well as active substances, and it is characterized by the presence of at least 10 chemical substances as active substances, among them:

allocriptonine (enantiomer) is present in the amount from 1.0 mg to 10.0 mg.,

nimodipine is present in the amount from 20.0 mg to 100.0 mg.,

potassium iodide is present in the amount from 120.0 mg to 560.0 mg.,

potassium iodate is present in the amount from 30.0 mg to 140.0 mg.,

inuline is present in the amount from 125.0 mg to 375.0 mg.,

silver is present in the amount from 0.10 mg to 0.50 mg.,

zinc is present in the amount from 10.0 mg to 20.0 mg.,

chromium is present in the amount from 0.05 mg to 0.20 mg.,

orotic acid is present in the amount from 150.0 mg to 500.0 mg.,

desferrine is present in the amount from 100.0 mg to 300.0 mg

It is beneficial when:

allocriptonine content is 10.0 mg.,

and nimodipine content is 100.0 mg.,

and potassium iodide content is 560.0 mg.,

and potassium iodate content is 140.0 mg.

and inuline content is 375.0 mg.,

and silver content is 0.50 mg.,

and zinc content is 20.0 mg.,

and chromium content is 0.20 mg.,

and orotic acid content is 500.0 mg.,

and desferrine content is 300.0 mg.

It is beneficial when:

allocriptonine content is 4.50 mg.,

and nimodipine content is 60.0 mg.,

and potassium iodide content is 340.0 mg.,

and potassium iodate content is 85.0 mg., and

inuline content is 250.0 mg., and

silver content is 0.30 mg.,

and zinc content is 15.0 mg.,

and chromium content is 0.125 mg.,

and orotic acid content is 325.0 mg.,

and desferrine content is 200.0 mg.

It is beneficial when:

allocriptonine content is 1.0 mg.,

and nimodipine content is 20.0 mg.,

and potassium iodide content is 120.0 mg.,

and potassium iodate content is 30.0 mg.,

and muline content is 125.0 mg.,

and silver content is 0.10 mg.,

and zinc content is 10.0 mg.,

and chromium content is 0.05 mg.,

and orotic acid content is 250.0 mg.,

and desferrine content is 100.0 mg.

It is beneficial when the weight ratio of allocriptonine to nimodipine to potassium iodide to potassium iodate to inuline to silver to zinc to chromium to orotic acid to desferrine is generally maintain as 1:20:120:30:125:0.1:10:0.05:250:100.

It is beneficial when the mixture of the ingredients has a form of a tablet, or coated tablet, or wafer, or suppository, or effervescent powder, or gel, or colloid solution.

It is beneficial when the mixture of the ingredients has a form of suspension, or syrup, or salt soluble in body fluids, or liquids for intramuscular or IV injections.

The production of the pharmaceutical mixture for blocking the HIV-1 and HIV-2 virus replication in the CD4+ cells of the human immune system in all stages of that viral infection, and in AIDS, is characterized by by mixing, in the room temperature, the following ingredients:—allocriptonine (enantiomer) in the amount from 1.0 mg to 10.0 mg.,

nimodipme in the amount from 20.0 mg to 100.0 mg.,

potassium iodide in the amount from 120.0 mg to 560.0 mg.,

potassium iodate in the amount from 30.0 mg to 140.0 mg.,

inuline in the amount from 125.0 mg to 375.0 mg.,

silver in the amount from 0.10 mg to 0.50 mg.,

zinc in the amount from 10.0 mg to 20.0 mg.,

chromium in the amount from 0.05 mg to 0.20 mg.,

orotic acid in the amount from 150.0 mg to 500.0 mg.,

desferrine in the amount from 100.0 mg to 300.0 mg, until homogeneous mixture is created.

It is beneficial when:

allocriptonine content is 10.0 mg.,

and nimodipine content is 100.0 mg.,

and potassium iodide content is 560.0 mg.,

and potassium iodate content is 140.0 mg.,

and inuline content is 375.0 mg.,

and silver content is 0.50 mg.,

and zinc content is 20.0 mg.,

and chromium content is 0.20 mg.,

and orotic acid content is 500.0 mg.,

and desferrine content is 300.0 mg.

It is beneficial when:

allocriptonine content is 4.50 mg.,

and nimodipine content is 60.0 mg.,

and potassium iodide content is 340.0 mg.,

and potassium iodate content is 85.0 mg.,

and inuline content is 250.0 mg.,

and silver content is 0.30 mg.,

and zinc content is 15.0 mg.,

and chromium content is 0.125 mg.,

and erotic acid content is 325.0 mg.,

and desferrine content is 200.0 mg.

It is beneficial when:

allocriptonine content is 1.0 mg.,

and nimodipine content is 20.0 mg.,

and potassium iodide content is 120.0 mg.,

and potassium iodate content is 30.0 mg.,

and inuline content is 125.0 mg.,

and silver content is 0.10 mg.,

and zinc content is 10.0 mg.,

and chromium content is 0.05 mg.,

and orotic acid content is 250.0 mg.,

and desferrine content is 100.0 mg.

It is beneficial when the weight ratio of allocriptonine to nimodipme to potassium iodide to potassium iodate to inuline to silver to zinc to chromium to orotic acid to desferrine is generally maintain as 1:20:120:30:125:0.1:10:0.05:250:100.

It is beneficial when the mixture of the ingredients has a form of a tablet, or coated tablet, or wafer, or suppository, or effervescent powder, or gel, or a colloid solution.

It is beneficial when the mixture of the ingredients has a form of suspension, or syrup, or salt soluble in body fluids, or liquids for intramuscular or IV injections.

The beneficial effect of the pharmaceutical mixture in the invention is blocking the mechanism of attaching the gp 160 protein of the HIV virus capsule to the receptors of the CD4+ cells of the human immune system, as well as blocking the intracellular processes related to the HIV-1 and HIV-2 virus replication. Example No. 1. Content of the pharmaceutical mixture:

allocriptonine (enantiomer) in the amount of 10.0 mg.,

nimodipine in the amount of 100.0 mg.,

potassium iodide in the amount of 560.0 mg.,

potassium iodate in the amount of 140.0 mg.,

inuline in the amount of 375.0 mg.,

silver in the amount of 0.50 mg.,

zinc in the amount of 20.0 mg.,

chromium in the amount of 0.20 mg.,

orotic acid in the amount of 500.0 mg.,

desferrine in the amount of 300.0 mg.

Production of the Pharmaceutical Mixture:

Mix together in the room temperature: 10.0 mg. of enantiomer of allocriptonine, 100.0 mg. of nimodipine, 560.0 mg. of potassium iodide, 140.0 mg of potassium iodate, 375.0 mg. of inuline, 0.50 mg of silver, 20.0 mg. of zinc, 0.20 mg. of chromium, 500.0 mg. of orotic acid, and 300.0 mg. of desferrine.

After adding all ingredients, mix them all together for about 10-20 minutes until a uniform substance is produced. The mix can then be pressed into tablets, and can also have a form of coated tablets, or can be shaped into a wafer, a suppository, effervescent powder, gel, or colloid solution.

### EXAMPLE NO. 2

### Content of the Pharmaceutical Mixture

allocriptonine (enantiomer) in the amount of 1.0 mg.,

nimodipine in the amount of 20.0 mg.,

potassium iodide in the amount of 120.0 mg.,

potassium iodate in the amount of 30.0 mg.,

mulme in the amount of 125.0 mg.,

silver in the amount of 0.10 mg.,

zinc in the amount of 10.0 mg.,

chromium in the amount of 0.05 mg.,

orotic acid in the amount of 150.0 mg.,

desferrine in the amount of 100.0 mg.

Production of the Pharmaceutical Mixture:

Mix together in the room temperature: 1.0 mg. of enantiomer of allocriptonine, 20.0 mg. of nimodipine, 120.0 mg. of potassium iodide, 30.0 mg of potassium iodate, 125.0 mg. of inuline, 0.10 mg of silver, 10.0 mg. of zinc, 0.05 mg. of chromium, 150.0 mg. of erotic acid, and 100.0 mg. of desferrine.

After adding all ingredients, mix them all together for about 10-20 minutes until a uniform substance is produced.

The product can then be made into suspension, syrup, alt soluble in body fluids, or liquid for intramuscular or IV injections, also in ampules.

It should be understood that the term “pharmaceutically effective amount” means an effective amount of the pharmaceutical mixture of the present invention (comprising a mixture of allocriptonine, nimodipine, potassium iodide, potassium iodate, inuline, silver, zinc, chromium, orotic acid and desferrine). Actual dosage levels of the pharmaceutical composition of this invention can be varied so as to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

We claim:

1. A therapeutic composition for the treatment of HIV in a patient in need of such treatment, the therapeutic composition comprising of pharmaceutically effective amounts of allocriptonine enantiomer, nimodipine, potassium iodide, potassium iodate, inuline, silver, zinc, chromium, orotic acid and desferrine.

2. The therapeutic composition of claim 1, wherein the allocriptonine enantiomer is present in the amount from about 1.0 mg to about 10.0 mg, nimodipine is present in the amount from about 20.0 mg to about 100.0 mg, potassium iodide is present in the amount from about 120.0 mg to about 560.0 mg, potassium iodate is present in the amount from about 30.0 mg to about 140.0 mg, inuline is present in the amount from about 125.0 mg to about 375.0 mg, silver is present in the amount from about 0.10 mg to about 0.50 mg, zinc, present in the amount from 10.0 mg to about 20.0 mg, chromium is present in the amount from about 0.05 mg to about 0.20 mg, orotic acid is present in the amount from about 150.0 mg to about 500.0 mg, and the desferrine is present in the amount from about 100.0 mg to about 300.0 mg.

3. The therapeutic composition of claim 1, wherein the allocriptonine enantiomer is present in the amount of about 10.0 mg, the nimodipine is present in the amount of about 100.0 mg, the potassium iodide is present in the amount of about 560.0 mg, the potassium iodate is present in the amount of about 140.0 mg, the inuline is present in the amount of about 375.0 mg, the silver is present in the amount of about 0.50 mg, the zinc is present in the amount of about 20.0 mg, the chromium is present in the amount of about 0.20 mg, the orotic acid is present in the amount of about 500.0 mg, and the desferrine is present in the amount of about 300.0 mg.

4. The therapeutic composition of claim 1, wherein the allocriptonine enantiomer is present in the amount of about 4.5 mg, the nimodipine is present in the amount of about 60.0 mg, the potassium iodide is present in the amount of about 340.0 mg, the potassium iodate is present in the amount of about 85.0 mg, the inuline is present in the amount of about 250.0 mg, the silver is present in the amount of about 0.30 mg, the zinc is present in the amount of about 15.0 mg, the chromium is present in the amount of about 0.125 mg, the orotic acid is present in the amount of about 325.0 mg, and the desferrine is present in the amount of about 200.0 mg.

5. The therapeutic composition of claim 1, wherein the allocriptonine enantiomer is present in the amount of about 1.0 mg, the nimodipine is present in the amount of about 20.0 mg, the potassium iodide is present in the amount of about 120.0 mg, the potassium iodate is present in the amount of about 30.0 mg, the inuline is present in the amount of about 125.0 mg, the silver is present in the amount of about 0.10 mg, the zinc is present in the amount of about 10.0 mg, the chromium is present in the amount of about 0.05 mg, the orotic acid is present in the amount of about 250.0 mg, and the desferrine is present in the amount of about 100.0 mg.

6. The therapeutic composition of claim 1 further comprises a weight ratio of allocriptonine to nimodipine to potassium iodide to potassium iodate to inuline to silver to zinc to chromium to orotic acid to desferrine of about 1:20:120:30:125:0.1:10:0.05:250:100, respectively.

7. The therapeutic composition of claim 1, wherein said composition is in a form suitable for administering to a patient, wherein the form is selected from the group consisting of a tablet, coated tablet, wafer, suppository, effervescent powder, gel, and a colloid suspension.

8. A method of preparing a pharmaceutical mixture for blocking HIV virus replication, comprising the step of mixing together pharmaceutically effective amounts of allocriptonine (enantiomer), nimodipine, potassium iodide, potassium iodate, inuline, silver, zinc, chromium, orotic acid, and desferrine to provide the pharmaceutical mixture for blocking HIV-1 and HIV-2 virus replication.

9. The method of claim 8, wherein the step of mixing is performed at room temperature.

10. The method of claim 8, wherein the allocriptonine enantiomer is present in the amount from about 1.0 mg to about 10.0 mg, nimodipine is present in the amount from about 20.0 mg to about 100.0 mg, potassium iodide is present in the amount from about 120.0 mg to about 560.0 mg, potassium iodate is present in the amount from about 30.0 mg to about 140.0 mg, inuline is present in the amount from about 125.0 mg to about 375.0 mg, silver is present in the amount from about 0.10 mg to about 0.50 mg, zinc, present in the amount from 10.0 mg to about 20.0 mg, chromium is present in the amount from about 0.05 mg to about 0.20 mg, orotic acid is present in the amount from about 150.0 mg to about 500.0 mg, and the desferrine is present in the amount from about 100.0 mg to about 300.0 mg.

11. The method of claim 8, wherein the allocriptonine enantiomer is present in the amount of about 10.0 mg, the nimodipine is present in the amount of about 100.0 mg, the potassium iodide is present in the amount of about 560.0 mg, the potassium iodate is present in the amount of about 140.0 mg, the inuline is present in the amount of about 375.0 mg, the silver is present in the amount of about 0.50 mg, the zinc is present in the amount of about 20.0 mg, the chromium is present in the amount of about 0.20 mg, the orotic acid is present in the amount of about 500.0 mg, and the desferrine is present in the amount of about 300.0 mg.

12. The method of claim 8, wherein the allocriptonine enantiomer is present in the amount of about 4.5 mg, the nimodipine is present in the amount of about 60.0 mg, the potassium iodide is present in the amount of about 340.0 mg, the potassium iodate is present in the amount of about 85.0 mg, the inuline is present in the amount of about 250.0 mg, the silver is present in the amount of about 0.30 mg, the zinc is present in the amount of about 15.0 mg, the chromium is present in the amount of about 0.125 mg, the orotic acid is present in the amount of about 325.0 mg, and the desferrine is present in the amount of about 200.0 mg.

13. The method of claim 8, wherein the allocriptonine enantiomer is present in the amount of about 1.0 mg, the nimodipine is present in the amount of about 20.0 mg, the potassium iodide is present in the amount of about 120.0 mg, the potassium iodate is present in the amount of about 30.0 mg, the inuline is present in the amount of about 125.0 mg, the silver is present in the amount of about 0.10 mg, the zinc is present in the amount of about 10.0 mg, the chromium is present in the amount of about 0.05 mg, the orotic acid is present in the amount of about 250.0 mg, and the desferrine is present in the amount of about 100.0 mg.

14. The method of claim 8, wherein the pharmaceutical mixture comprises a weight ratio of allocriptonine to nimodipine to potassium iodide to potassium iodate to inuline to silver to zinc to chromium to orotic acid to desferrine of about 1:20:120:30:125:0.1:10:0.05:250:100, respectively.

15. The method of claim 8 further comprising converting the pharmaceutical mixture into a form suitable for administering to a patient, wherein the form is selected from the group consisting of a tablet, coated tablet, wafer, suppository, effervescent powder, gel, and a colloid.

16. A method of treating a HIV infection, the method comprising administering to a patient in need of such treatment a pharmaceutical mixture comprising of pharmaceutically effective amounts of allocriptonine enantiomer, nimodipine, potassium iodide, potassium iodate, inuline, silver, zinc, chromium, orotic acid and desferrine.

17. The method of claim 16, wherein the pharmaceutical mixture is administered intramuscularly.

18. The method of claim 16, wherein the pharmaceutical mixture is administered by intravenous injection.
